(19) United States
(12) Patent Application Publication
(10) Pub. No.: US 2016/0157858 A1
(43) Pub. Date: Jun. 9, 2016

(54) DOUBLE BARBED SUTURE WITH NEEDLE DELIVERY SYSTEM

(71) Applicant: BOSTON SCIENTIFIC SCIMED, INC., Maple Grove, MN (US)

(72) Inventors: Melissa Joan HORTON, Watertown, MA (US); William Bertolino, Framingham, MA (US); Heather A. Simani, Dedham, MA (US); Jennifer Marie Mehl, Watertown, MA (US); Polihronis Vasiliadis, Worcester, MA (US); Todd Mannion Pfizenmaier, Franklin, MA (US); Peter L. Dayton, Brookline, MA (US)

(21) Appl. No.: 14/943,708

(22) Filed: Nov. 17, 2015

Related U.S. Application Data
(60) Provisional application No. 62/086,919, filed on Dec. 3, 2014.

(51) Int. Cl. A61B 17/06; A61B 17/04

(52) U.S. Cl. CPC A61B 17/06066; A61B 17/0469; A61B 2017/00407

(57) ABSTRACT

A tissue closure device includes a suture extending longitudinally from a proximal end to a distal end; a distal barb element including at least one distal arm extending from a first end connected to the distal end of the suture to a second end extending toward the proximal end of the suture, the distal barb movable between an engaging configuration, in which the distal arm extends away from the suture; to a non-engaging configuration in which the distal arm is moved toward the suture; and a proximal barb element slidably mounted over the suture and including a proximal arm extending from a first end connected to a base portion mounted over the suture to a second extending toward the distal end of the suture, the proximal barb element movable between an engaging configuration and a non-engaging configuration.

### PRIORITY CLAIM

This application claims priority to U.S. Provisional Patent Application Serial No. 62/086,919 filed Dec. 3, 2014; the disclosure of which is incorporated herewith by reference.

### BACKGROUND

Physicians have become more willing to perform more aggressive interventional and therapeutic endoscopic procedures including, for example, removal of larger lesions (e.g., cancerous masses), tunneling under mucosal layers in the gastro-intestinal (GI) tract to treat tissues below the mucosa, full thickness removal of tissue, inserting devices through the GI tract and then penetrating the GI organ to treat tissue outside the GI tract, and endoscopic treatment/repair of post-surgical issues (e.g., post-surgical leaks, breakdown of surgical staple lines, anastomotic leaks). These procedures may increase the risk of perforating the wall of the GI tract, or may require closure of the GI tract wall as part of the procedure. Endoscopic closure reduces cost and may reduce the trauma and inconvenience associated with these procedures. However, conventional tissue closure devices may be insufficient to close certain perforations.

### SUMMARY

The present disclosure is directed to a tissue closure device, comprising a suture extending longitudinally from a proximal end to a distal end, a distal barb element including at least one distal arm extending from a first end connected to the distal end of the suture to a second end extending toward the proximal end of the suture, the distal barb movable between an engaging configuration, in which the distal arm extends away from the suture, to a non-engaging configuration in which the distal arm is moved toward the suture, and a proximal barb element slidably mounted over the suture proximally of the distal barb element, the proximal barb element including a proximal arm extending from a first end connected to a base portion mounted over the suture to a second end extending toward the distal end of the suture, the proximal barb element movable between an engaging configuration, in which the proximal arm extends away from the suture, and a non-engaging configuration, in which the proximal arm is moved toward the suture.

In an embodiment, the distal barb element may be biased in the engaging configuration.

In an embodiment, the proximal barb element may be biased in the engaging configuration.

In an embodiment, a portion of an exterior of the suture and an interior of the base portion of the proximal barb element may include corresponding ratchet teeth such that the proximal barb element is permitted to be moved distally relative to the suture prevented from being moved proximally relative to the suture.

In an embodiment, one of the suture, distal barb element and the proximal barb element may be comprised of a biodegradable material.

In an embodiment, the suture may be formed of a heat activated material which coils upon being heated to a predetermined temperature.

In an embodiment, the distal end of the suture may include a distal locking feature and the proximal end of the suture includes a proximal locking feature.

The present disclosure is also directed to a system for treating a tissue defect, comprising a plurality of tissue closure devices longitudinally connectable to one another. Each of the plurality of tissue closure devices includes a suture extending longitudinally from a proximal end to a distal end, a distal barb element including at least one distal arm extending from a first end connected to the distal end of the suture to a second end extending toward the proximal end of the suture, the distal barb movable between an engaging configuration, in which the distal arm extends away from the suture, to a non-engaging configuration in which the distal arm is moved toward the suture, and a proximal barb element slidably mounted over the suture proximally of the distal barb element, the proximal barb element including a proximal arm extending from a first end connected to a base portion mounted over the suture to a second end extending toward the distal end of the suture, the proximal barb element movable between an engaging configuration, in which the proximal arm extends away from the suture, and a non-engaging configuration, in which the proximal arm is moved toward the suture. The system also comprises a delivery tool including a needle extending longitudinally from a proximal end to a distal end and including a lumen extending therethrough, the plurality of tissue closure devices housed within the lumen of the needle in longitudinal alignment.

In an embodiment, each of the tissue closure devices may include a distal locking feature at the distal end of the suture and a proximal locking feature at the proximal end of the suture such that distal and proximal locking features of adjacent ones of the plurality of tissue closure devices engage one another to lock the adjacent tissue closure devices relative to one another.

In an embodiment, the delivery tool may further include a pusher slidably received within the lumen of the needle, the delivery tool extending longitudinally from a proximal end to a distal end and including a lumen extending therethrough, the pusher extending over the plurality of tissue closure devices to provide radial compression to the distal and proximal locking features of the adjacent tissue closure devices to lock the adjacent tissue closure devices relative to one another.

In an embodiment, the system may further comprise a handle portion including actuators for moving each of the plurality of tissue closure devices, the pusher and the needle relative to one another.

In an embodiment, the needle may include a tapered distal tip for piercing a target tissue defect to be treated.

In an embodiment, a portion of an exterior of the suture and an interior of the base portion of the proximal barb element may include corresponding ratchet teeth such that the proximal barb element is permitted to be moved distally relative to the suture prevented from being moved proximally relative to the suture.

In an embodiment, one of the suture, distal barb element and the proximal barb element may be comprised of a biodegradable material.

In an embodiment, the suture may be formed of a heat activated material which coils upon being heated to a predetermined temperature.

The present disclosure is also directed to a method for closing a tissue defect, comprising inserting a plurality of tissue closure devices slidably housed within a delivery tool, in an insertion configuration, to a target tissue defect to be treated within a patient body, the plurality of tissue closure devices in longitudinal alignment relative to one another, piercing the portions of tissue on a first and second opposing side of the tissue defect with the needle such that the needle extends across the tissue defect and a distal end of the needle is distal of the first side and the proximal end of the needle is proximal of the second side, moving the plurality of tissue closure devices distally relative to the needle so that a distal barb element of the distal-most one of the plurality of tissue closure devices is moved distally past the distal end of the needle to engage the portion of tissue on the first side of the tissue defect, moving the needle proximally relative to the plurality of tissue closure devices so that the distal end of the needle is moved proximally past a proximal barb element of the distal-most tissue closure device such that the proximal barb element of the distal-most tissue closure device engages the portion of tissue along the second side of the tissue closure device, and sliding the proximal barb element of the distal-most tissue closure device distally along a longitudinally extending suture of the distal-most tissue closure device toward the distal barb element thereof to draw the first and second sides of the tissue defect toward one another.

### BRIEF DESCRIPTION

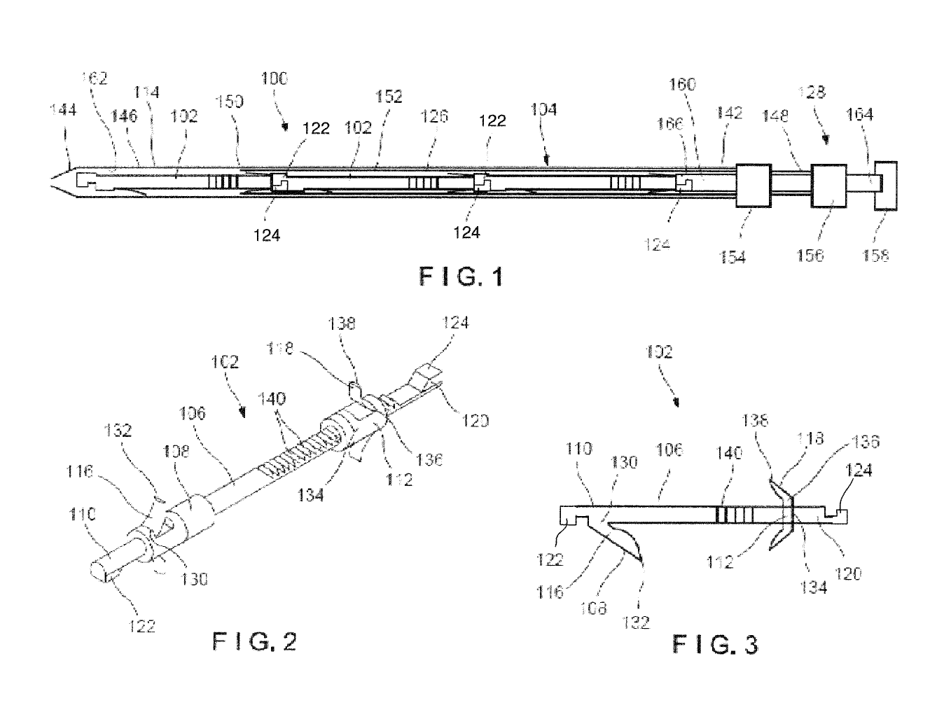

FIG. 1 illustrates a longitudinal cross-sectional view of a system according to an exemplary embodiment of the present disclosure; and

FIG. 2 illustrates a perspective view of a tissue closure device of the system of FIG. 1; and

FIG. 3 illustrates a longitudinal side view of the tissue closure device of the system of FIG. 1.

### DETAILED DESCRIPTION

The present disclosure may be further understood with reference to the following description and the appended drawings, wherein like elements are referred to with the same reference numerals. The present disclosure relates to devices for tissue closure and, in particular, to an endoscopic tissue closure device. Exemplary embodiments of the present disclosure describe a tissue closure device comprising a suture including a distal barb element for engaging a portion of tissue on a first side of a tissue defect to be treated and a proximal barb element for engaging a portion on a second side of the tissue defect opposite the first side. The distal and proximal barb elements are movable relative to one another along a length of the suture such that moving the distal and proximal barb elements toward one another closes the tissue defect. Multiple tissue closure devices may be delivered to the tissue defect, as necessary, via a needle in which the tissue closure devices are housed. It will be understood by those of skill in the art that the terms “proximal” and “distal,” as used herein, are intended to refer to a direction toward (proximal) and away from (distal) a user of the device.

As shown in FIGS. 1 - 2, a system 100 according to an exemplary embodiment of the present disclosure comprises a plurality of tissue closure devices 102 deliverable to a target tissue via a delivery tool 104 including a needle 114. Each of the tissue closure devices 102 includes a suture 106 along with a distal barb element 108 at a distal end 110 thereof and a proximal barb element 112 movably mounted over the suture 106 proximally of the distal barb element 108. The distal barb element 108 may include one or more arms 116 for engaging a portion of tissue along a first side of a tissue defect (e.g. tissue opening or perforation) while the proximal barb element 112 may include one or more arms 118 for engaging tissue along a portion of tissue along a second side of the tissue defect opposite the first side so that when the proximal barb element 112 is moved distally toward the distal barb element 108, the edges of the tissue opening are drawn together toward a closed position. The tissue closure devices 102 are longitudinally aligned within the needle 114 of the delivery tool 104 so that each tissue closure device 102 may be individually deployed as needed. As will be described in further detail below, the delivery tool 108 further includes a pusher 126 extending over the plurality of tissue closure devices 102 in the needle 114 for locking the tissue closure devices 102 in longitudinal alignment relative to one another and for pushing the proximal distal barb element 112 of a distal-most tissue closure device 102 along a corresponding suture 106 thereof. The delivery tool 104 according to this embodiment also includes a handle portion 128 including actuators for moving the tissue closure devices 102, the pusher 126 and the needle 114 relative to one another during insertion and deployment o f the tissue closure devices 102 in the tissue defect.

The suture 106 of each of the tissue closure devices 102 extends longitudinally from a proximal end 120 to the distal end 110. The distal end 110 includes a distal locking feature 122 while the proximal end 120 includes a proximal locking feature 124. The distal and proximal locking features 122, 124 of longitudinally adjacent tissue closure devices 102 engage one another to lock the adjacent tissue closure devices 102 in longitudinal alignment relative to one another. The distal and proximal locking features 122, 124 of adjacent tissue closure devices 102 lock together in a lock and key fashion via radial compression applied. The pusher 126 of the delivery tool 104 extends thereover to lock the distal and proximal locking features to one another. Thus, adjacent tissue closure devices 102 remain locked relative to one another so long as the pusher 126 extends over the distal and proximal locking features 122, 124 thereof.

The distal barb element 108 of each of the tissue closure devices 102 includes one or more arms 116 extending from a first end 130 connected to the suture 106 to a second end 132 extending toward the proximal end 120 of the suture 106. The arms 116 of the distal barb element 108 are movable between an engaging configuration, in which the second end 132 of the arms 116 are separated from the suture 106, and a non-engaging configuration, in which the second end 132 is moved toward the suture 106. The arms 116 are biased toward the engaging configuration, but constrained in the non-engaging configuration via an interior surface of the needle 114 until the tissue closure device 102 is deployed therefrom. The arms 116 extend toward the proximal end 120 of the suture 106 so that the distal barb element 108 is prevented from being pulled proximally from a portion of tissue into which it has been inserted. The arms 116 may be integrally formed with or affixed to the suture 106 via any known fixation means. For example, the arms 116 may be affixed to the suture 106 via pressure from compression and/or affixed via heat.

The proximal barb element 112 of each of the tissue closure devices 102 includes a base portion 134 slidably mounted over the suture 106 and one or more arms 118 extending from the base portion 134. The arms 118 extend from a first end 136 connected to the base portion 134 to a second end 138 extending toward the distal end 110 of the suture 106. Similarly to the arms 116 of the distal barb element 108, the arms 118 of the proximal barb element 112 are movable between an engaging configuration, in which the second end 138 of the arms 118 extend away from the suture 106, and a non-engaging configuration, in which the second end 138 is moved toward the suture 106. The arms 118 may be biased toward the engaging configuration, but constrained via the interior surface of the needle 114 until the tissue closure device 102 is deployed from the needle 114 of the delivery tool 104. The arms 118 of the proximal barb element 112 extend toward the distal end 110 of the suture 106 so that proximal barb element 112 is prevented from being pulled distally from a portion of tissue into which it has been inserted. The arms 118 may be integrally formed with the base portion 134 or, alternatively, affixed thereto via any of a variety of known fixation means.

As described above, the proximal barb element 112 is longitudinally movable along a length of the suture 106 over which it has been mounted. An interior surface of the base portion 134 and an exterior surface of the suture 106 includes corresponding ratchet teeth 140 so that the proximal barb 112 may be moved in a distal direction along the suture 106, but is prevented from being moved proximally therelaong. Thus, once the proximal barb element 112 has been moved toward the distal end 110 of the suture 106 to close the tissue defect, the tissue defect is held in the closed position. In one embodiment, the proximal barb element 112 may be disengagable from the ratchet teeth 140 by, for example, rotating the proximal barb element 112 about the suture 106 so that the base portion 134 may be unthreaded therefrom. A length of the suture 106 may be selected to correspond to a size of the tissue defect to be treated. The suture 106, distal barb element 108 and/or the proximal barb element 112 may be formed of plastic, metal and/or any other biodegradable material or a combination of these materials. Portions of the tissue closure device 102 may be formed via extrusion, molding and/or skiving technology as would be understood by those skilled in the art.

Although the exemplary embodiment specifically shows and describes ratchet teeth 140 for locking the proximal barb element 112 in a desired position along the suture 106, the tissue closure device 102 may include any locking or holding mechanism for securing the proximal barb element 102 in the desired position along the suture 106. For example, in another embodiment, the suture 106 may be formed of a heat activated material (e.g., pre-treated Nitinol or elastic material) which deforms to coil along a length thereof so that the proximal barb element 112 may be fixed to the proximal end 120 of the suture 106. Thus, when the tissue closure device 102 is deployed in the target tissue defect, the temperature of the patient's body will cause the length of the suture 106 to coil, drawing the distal and proximal barb elements 108, 112 toward one another to close the target tissue defect.

In another embodiment, the distal and proximal barb elements 108, 112 may be configured as radially expansible members extending over a portion of the suture 106. For example, the distal and proximal barb elements 108, 112 may be formed as knots along the suture 106, which expand to engage a tissue into which it is inserted.

The tissue closure devices 102 may be sequentially delivered to the tissue defect to be treated via the delivery tool 104, which is comprised of the needle 114, the pusher 126 and the handle portion 128 for moving the tissue closure devices 102, needle 114 and pusher 126 relative to one another. The needle 114 extends longitudinally from a proximal end 142 to a distal end 144 and includes a lumen 146 extending therethrough. As would be understood by those skilled in the art, the needle 114 may be formed of a material sufficiently flexible to be inserted to a target site in the body along a tortuous path (e.g., through a working channel of an endoscope extending within a body lumen). For example, the needle 114 may be formed of Nitinol. The distal end 144 may include a tapered tip to facilitate piercing of tissue thereby. The pusher 126 is slidably received within the lumen 146 and extends longitudinally from a proximal end 148 to a distal end 150. The pusher 126 also includes a lumen 152 extending therethrough.

The tissue closure devices 102 are received within the lumen 152 of the pusher 126 so that the pusher 126 extends over and provides radial compression to distal and proximal locking features 122, 124 between adjacent tissue closure devices 102 to lock the tissue closure devices 102 in longitudinal alignment with one another. In an insertion configuration, the pusher 126 extends over the proximal locking feature 124 of the distal-most one of the tissue closure devices 102 to lock the distal-most tissue closure device 102 to an immediately proximal tissue closure device 102, but does not extend over the distal and proximal barb elements 108, 112 of the distal-most tissue closure device 102. Thus, in the insertion configuration the distal and proximal barb elements 108, 112 of the distal-most tissue closure device 102 extend distally past the distal end 150 of the pusher 126 but proximally of the distal end 144 of the needle 114 so that the distal and proximal barb elements 108, 112 are constrained via an interior surface 162 of the needle 114 toward the non-engaging configurations.

The handle portion 128 may, for example, include a needle adjust 154 connected to the proximal end 142 of the needle 114, a pusher adjust 156 connected to the proximal end 148 of the pusher 126 and a closure device handle 158 connected to the proximal-most one of the tissue closure devices 102 via a connecting rod 160 extending through the lumen 152 of the pusher 126. The connecting rod 160 may extend longitudinally from a proximal end 164 connected to the closure handle 158 to a distal end 166 connected to the proximal locking feature 124 of the proximal-most one of the tissue closure devices 102. The needle adjust 154, the pusher adjust 156 and the closure device handle 158 are movable relative to one another to move the tissue closure devices 102, the needle 114 and the pusher 126 relative to one another during insertion and deployment of the tissue closure devices, as will be described in further detail below.

According to an exemplary surgical method using the system 100, the needle 114 of the delivery tool 104 is inserted, in the insertion configuration, into a patient's body to a position adjacent to the tissue defect to be treated via, for example, a working channel of an endoscope. The distal end 144 of the needle 114 is passed through portions of tissue along first and second opposing sides of the tissue defect so that the needle 114 extends across the tissue defect. In other words, the needle 114 pierces a first portion of tissue on a first side of the tissue defect (e.g., a proximal side of the tissue defect), and then extends across the tissue defect to pierce a second portion of tissue on a second side of the tissue defect opposite the first side (e.g., a distal side of the tissue defect). Once the needle 114 is extended across the tissue defect to pierce the first and second portions of tissue, the tissue closure devices 102 and the pusher 126 are moved distally relative to the needle 114 such that the distal barb element 108 of the distal-most one of the tissue closure devices 102 is moved distally past the distal 144 of the needle 114. As the distal barb element 108 is moved distally past the distal 144 of the needle 114, the arms 116 of the distal barb element 108 revert to the biased engaging configuration to engage the tissue along the first side of the tissue defect.

Once the arms 116 of the distal barb element 108 is engaged with tissue along the first side of the tissue defect, the needle 114 is drawn proximally relative to the tissue closure devices 102 via, for example, the needle adjust 154, to expose the proximal barb element 112 of the distal-most tissue closure device 102. As the distal end 144 of the needle 114 is drawn proximally past the proximal barb element 112, the arms 118 of the proximal barb element 112 are permitted to revert to the biased engaging configuration to engage tissue along the second side of the tissue opening. The pusher 126, which still extends over the proximal locking feature 124 of the distal-most tissue closure device 102, is then moved distally relative to the plurality of tissue closure devices 102 and the needle 114 to push the proximal locking feature 124 of the distal-most tissue closure device 102 distally along the suture 106 of the distal-most tissue closure device 102 to move the tissue defect toward a closed position.

The corresponding ratchet teeth 140 of the suture 106 and the proximal barb element 112 hold the tissue defect in the closed position by preventing the proximal barb element 112 from being moved proximally relative to the suture 106 once the proximal barb element 112 has been moved to a desired position along the suture 106. The pusher 126 is then drawn proximally relative to the plurality of tissue closure devices 102 and the needle 114 until the distal end 150 of the pusher is moved proximally past the proximal locking feature 124 of the distal-most tissue locking device 102 and a distal locking feature 122 of the immediately proximal tissue closure device 102, permitting the distal-most tissue closure device and the immediately proximal tissue closure device to disengage one another to release and deploy the distal-most tissue closure device 102. The pusher 126 may then be drawn further proximally relative to the remaining tissue closure devices 102 until the distal and proximal barb elements 108, 112 of the immediately proximal tissue closure device 102 are pushed out against the interior surface 126 of the needle 114 to be constrained thereby toward the non-engaging configuration. The above-described steps may then be repeated for the remaining tissue closure devices 102, as desired. In particular, subsequent tissue closure devices 102 may be deployed along a length of the tissue defect until the entire tissue defect has been closed.

In a further embodiment, the delivery tool 104 may be reloadable if additional tissue closure devices 102 are required to suture the tissue opening. Additional tissue closure devices 102 may be individually loaded or loaded via a cartridge housing a plurality of the tissue closure devices 102.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

1-15. (canceled)

16. A tissue closure device, comprising:
a suture extending longitudinally from a proximal end to a distal end;
a distal barb element including at least one distal arm extending from a first end connected to the distal end of the suture to a second end extending toward the proximal end of the suture, the distal barb movable between an engaging configuration, in which the distal arm extends away from the suture, to a non-engaging configuration in which the distal arm is moved toward the suture; and
a proximal barb element slidably mounted over the suture proximally of the distal barb element, the proximal barb element including a proximal arm extending from a first end connected to a base portion mounted over the suture to a second extending toward the distal end of the suture, the proximal barb element movable between an engaging configuration, in which the proximal arm extends away from the suture, and a non-engaging configuration, in which the proximal arm is moved toward the suture.

17. The device of claim 16, wherein the distal barb element is biased in the engaging configuration.

18. The device of claim 16, wherein the proximal barb element is biased in the engaging configuration.

19. The device of claim 16, wherein a portion of an exterior of the suture and an interior of the base portion of the proximal barb element include corresponding ratchet teeth such that the proximal barb element is permitted to be moved distally relative to the suture prevented from being moved proximally relative to the suture.

20. The device of claim 16, wherein one of the suture, distal barb element and the proximal barb element is comprised of a biodegradable material.

21. The device of claim 16, wherein the suture is formed of a heat activated material which coils upon being heated to a predetermined temperature.

22. The device of claim 16, wherein the distal end of the suture includes a distal locking feature and the proximal end of the suture includes a proximal locking feature.

23. A system for treating a tissue defect, comprising:
a plurality of tissue closure devices longitudinally connectable to one another, each of the plurality of tissue closure devices including:
a suture extending longitudinally from a proximal end to a distal end;
a distal barb element including at least one distal arm extending from a first end connected to the distal end of the suture to a second end extending toward the proximal end of the suture, the distal barb movable between an engaging configuration, in which the distal arm extends away from the suture, to a non-engaging configuration in which the distal arm is moved toward the suture; and
a proximal barb element slidably mounted over the suture proximally of the distal barb element, the proximal barb element including a proximal arm extending from a first end connected to a base portion mounted over the suture to a second extending toward the distal end of the suture, the proximal barb element movable between an engaging configuration, in which the proximal arm extends away from the suture, and a non-engaging configuration, in which the proximal arm is moved toward the suture; and
a delivery tool including a needle extending longitudinally from a proximal end to a distal end and including a lumen extending therethrough, the plurality of tissue closure devices housed within the lumen of the needle in longitudinal alignment.

24. The system of claim 23, wherein each of the tissue closure devices include a distal locking feature at the distal end of the suture and a proximal locking feature at the proximal end of the suture such that distal and proximal locking features of adjacent ones of the plurality of tissue closure devices engage one another to lock the adjacent tissue closure devices relative to one another.

25. The system of claim 24, the delivery tool further including a pusher slidably received within the lumen of the needle, the delivery tool extending longitudinally from a proximal end to a distal end and including a lumen extending therethrough, the pusher extending over the plurality of tissue closure devices to provide radial compression to the distal and proximal locking features of the adjacent tissue closure devices to lock the adjacent tissue closure devices relative to one another.

26. The system of claim 24, further comprising a handle portion including actuators for moving each of the plurality of tissue closure devices, the pusher and the needle relative to one another.

27. The system of claim 23, wherein the needle includes a tapered distal tip for piercing a target tissue defect to be treated.

28. The system of claim 23, wherein a portion of an exterior of the suture and an interior of the base portion of the proximal barb element include corresponding ratchet teeth such that the proximal barb element is permitted to be moved distally relative to the suture prevented from being moved proximally relative to the suture.

29. The system of claim 23, wherein one of the suture, distal barb element and the proximal barb element is comprised of a biodegradable material.

30. The system of claim 23, wherein the suture is formed of a heat activated material which coils upon being heated to a predetermined temperature.

31. A method for closing a tissue defect, comprising:
inserting a plurality of tissue closure devices slidably housed within a delivery tool, in an insertion configuration, to a target tissue defect to be treated within a patient body, the plurality of tissue closure devices in longitudinal alignment relative to one another;
piercing the portions of tissue on a first and second opposing side of the tissue defect with the needle such that the needle extends across the tissue defect and a distal end of the needle is distal of the first side and the proximal end of the needle is proximal of the second side;
moving the plurality of tissue closure devices distally relative to the needle so that a distal barb element of the distal-most one of the plurality of tissue closure devices is moved distally past the distal end of the needle to engage the portion of tissue on the first side of the tissue defect;
moving the needle proximally relative to the plurality of tissue closure devices so that the distal end of the needle is moved proximally past a proximal barb element of the distal-most tissue closure device such that the proximal barb element of the distal-most tissue closure device engages the portion of tissue along the second side of the tissue closure device; and
sliding the proximal barb element of the distal-most tissue closure device distally along a longitudinally extending suture of the distal-most tissue closure device toward the distal barb element thereof to draw the first and second sides of the tissue defect toward one another.

32. The method of claim 31, wherein the proximal barb element is moved distally along the suture via a pusher extending slidably through the needle and over the plurality of tissue closure devices, a distal end of the pusher positioned proximally of the proximal barb element of the distal-most tissue closure device.

33. The method of claim 32, wherein the adjacent tissue closure devices are locked in longitudinal alignment relative to one another via a radial compression of the pusher extending over corresponding locking features thereof.

34. The method of claim 33, further comprising drawing the pusher proximally relative to the plurality of tissue closure devices until the distal end of the pusher is moved proximally past the corresponding locking features between the distal-most tissue closure device and an immediately proximal tissue closure device, releasing the connection therebetween and deploying the distal-most tissue closure device in the patient body.

35. The method of claim 31, wherein each of the distal and proximal barb elements include an arm movable between an engaging configuration, in which the arms extend away from the suture, and a non-engaging configuration, in which the arms are moved toward the suture, the arms constrained toward the non-engaging configuration via an interior surface of the needle in the insertion configuration.
